(19) United States
(12) Patent Application Publication
(10) Pub. No.: US 2013/0041376 A1
(43) Pub. Date: Feb. 14, 2013

(54) PROSTHESIS RESECTION GUIDE

(71) Applicants: David J. Neal, Morris Plains, NJ (US); Jorge Montoya, Madison, NJ (US); John Chernosky, Brick, NJ (US); Ray Zubok, Midland Park, NJ (US); Keith A. Roby, Jersey City, NJ (US); Timothy A. Hoeman, Morris Plains, NJ (US)

(72) Inventors: David J. Neal, Morris Plains, NJ (US); Jorge Montoya, Madison, NJ (US); John Chernosky, Brick, NJ (US); Ray Zubok, Midland Park, NJ (US); Keith A. Roby, Jersey City, NJ (US); Timothy A. Hoeman, Morris Plains, NJ (US)

(73) Assignee: Zimmer, Inc., Warsaw, IN (US)

(21) Appl. No.: 13/570,311

(22) Filed: Aug. 9, 2012

Related U.S. Application Data
(60) Provisional application No. 61/522,872, filed on Aug. 12, 2011.

(51) Int. Cl. A61B 17/17

(52) U.S. Cl. 606/79; 606/88

(57) ABSTRACT

A conical burr template is indexed to the intramedullary canal of a tibia, such that a burr may trace the periphery of the template to define a correspondingly shaped cavity in the bone which is properly sized, shaped, and positioned to receive a cone-shaped tibial augment component. Advantageously, use of the burr template promotes expedient surgery while maintaining optimal fit characteristics and proper spatial placement of the tibial cone augment.

### CLAIM OF PRIORITY

This application claims the benefit of priority under 35 U.S.C. §119(e) of Neal et al., U.S. Provisional Patent Application Ser. No. 61/522,872, entitled “PROSTHESIS RESECTION GUIDE”, filed on Aug. 12, 2011, which is herein incorporated by reference in its entirety.

### BACKGROUND

1. Technical Field

The present disclosure relates to orthopedic prostheses, more particularly, to guides for resecting bone in preparation to receive a prosthetic component.

2. Description of the Related Art

Orthopedic prostheses are commonly utilized to prepare and/or replace damaged bone and tissue in the human body. For example, a knee prosthesis may be used to restore natural knee function by repairing damaged or diseased articular surfaces of the femur and/or tibia. Knee prostheses may include a femoral component implanted on the distal end of a femur, which articulates with a tibial component implanted on the corresponding proximal end of tibia. The femoral and tibial components cooperate to restore the function of healthy natural knee.

In some cases, the proximal tibia or distal femur may exhibit severe degeneration, trauma, or other pathologies, necessitating resection of more bone than can be compensated for by traditional femoral and tibial components. In such cases, augments may be used to effectively increase the size of an implanted component, thereby compensating for the additional volume of resected bone.

In the proximal tibia, for example, poor quality bone stock may exist around the medullary canal in the diaphyseal or metaphyseal region of the bone. In such cases, an augment having a generally truncated cone-shaped outer profile corresponding to typically cone-shaped bone defect encountered around the medullary canal may be used. Such tibial cone augments may be used in order to mimic the exterior periphery of the natural bone and thereby limit resection of healthy bone stock. To this end, cone-shaped augments may define irregular conical shapes, such as shapes having differing tapers at the medial and lateral sides versus the anterior and posterior sides. Further, tibial cone augments may define a generally oval cross section, which accommodates the natural proximal tibial geometry having greater width in a medial-lateral direction compared to the anterior-posterior direction.

Other indications for prosthetic implant augments include revision surgeries, in which formerly implanted prosthetic components are removed together with surrounding bone stock and replaced with new components. In such revision surgeries, the total volume of bone stock removed may be substantially greater than the bone stock removed during primary procedure, i.e., a procedure in which a first prosthesis is implanted to replace natural articular surfaces.

Exemplary tibial cone augments are disclosed in U.S. patent application Ser. No. 11/560,276, filed Nov. 15, 2006 and entitled “PROSTHETIC IMPLANT SUPPORT STRUCTURE,” and in U.S. patent application Ser. No. 12/886,297, filed Sep. 20, 2010 and entitled “TIBIAL AUGMENTS FOR USE WITH KNEE JOINT PROSTHESES, METHOD OF IMPLANTING THE TIBIAL AUGMENT, AND ASSOCIATED TOOLS,” and in U.S. Provisional Patent Application Ser. No. 61/488,549, filed May 20, 2011 and entitled “STABILIZING PROSTHESIS SUPPORT STRUCTURE,” all of which are commonly assigned with the present application, the entire disclosures of which are hereby expressly incorporated by reference herein.

In preparation for implantation of cone-shaped augment, a correspondingly cone-shaped cavity is formed in the bone. Instruments which aid in the expedient and accurate creation of this cavity have been the focus of substantial design efforts, particularly for the irregular cavities created for modern cone-shaped augments.

### SUMMARY

The present disclosure provides conical burr template which is indexed to the intramedullary canal of a tibia, such that a burr may trace the periphery of the template to define correspondingly shaped cavity in the bone which is properly sized, shaped, and positioned to receive a cone-shaped tibial augment component. Advantageously, use of the burr template promotes expedient surgery while maintaining optimal fit characteristics and proper spatial placement of the tibial cone augment.

The burr template mounts directly to an intramedullary rod used in other aspects of a knee implantation procedure, such that the cone-shaped cavity created by use of the burr template references the intramedullary canal. Advantageously, because other instruments and the prosthetic components may also reference the intramedullary canal a resection cavity defined by the template facilitates proper placement and orientation of the final implanted prosthesis. Further, the present burr template provides an expedient and accurate guide for creating a bone resection with highly precise and complex geometrical configurations to provide an ideal match with the size of a given augment component.

In one form thereof, the present disclosure provides a burr template comprising: a template track comprising a lateral track portion, a posterior track portion, and a medial track portion; a coupler joining the lateral track portion and the medial track portion, the coupler having a bore formed therethrough; and the bore spaced from the lateral track portion and the medial track portion, such that the template track defines an inner periphery corresponding to an outer periphery of tibial cone augment, the inner periphery adapted to be indexed to an intramedullary canal of a tibia.

In one form thereof, the present disclosure provides a method for resecting cavity in a bone, comprising: inserting an intramedullary rod into an intramedullary canal of the bone; passing a template over the intramedullary rod and coupling the template to the intramedullary rod adjacent the bone, the template having a template track extending away from the coupler; and sweeping a cutting instrument around the template track to define an inner periphery of the resection void.

### BRIEF DESCRIPTION OF THE DRAWINGS

The above-mentioned and other features and advantages of this disclosure, and the manner of attaining them, will become more apparent and the invention itself will be better understood by reference to the following descriptions of embodiments of the invention taken in conjunction with the accompanying drawings, wherein:

FIG. 1 is a perspective view of a conical burr template in accordance with the present disclosure, illustrated adjacent a proximal tibia;

FIG. 2 is a perspective view of the template shown in FIG. 1, illustrating assembly of an alignment bushing to the template;

FIG. 3A is a top plan view of a centered alignment bushing in accordance with the present disclosure;

FIG. 3B is a top plan view of an offset alignment bushing in accordance with the present disclosure;

FIG. 4 is a sagittal, elevation view of the template and bushing shown in FIG. 2, illustrating assembly thereof to a tibia with a positive anteroposterior slope;

FIG. 5 is a perspective view of the template and bushing shown in FIG. 2, illustrating securement of the bushing to the template;

FIG. 6 is a perspective view of the template and bushing shown in FIG. 2, together with a securement mechanism for securing the template to the tibia;

FIG. 7 is a perspective view of the template, bushing, and securement mechanism shown in FIG. 6, illustrating attachment of the securement mechanism to the template;

FIG. 8 is an exploded, perspective view of burr guard and burr in accordance with the present disclosure;

FIG. 9 is a perspective view of the template, bushing, and securement mechanism shown in FIG. 6, together with the burr guard and burr shown in FIG. 8;

FIG. 10 is a perspective view of the tibia shown in FIG. 1 after an initial resection, illustrating use of tibial cone augment to prepare for further resection

FIG. 11A is a perspective view of the tibia shown in FIG. 10, illustrating further resection thereof using a burr sleeve;

FIG. 11B is a side, elevation view of the tibia shown in FIG. 11A after completion of a cone shaped resection;

FIG. 12 is a perspective view of the tibia shown in FIG. 11A after completion of a cone shaped resection, illustrating implantation of a tibial cone augment;

FIG. 13 is a perspective view of an alternative conical burr template made in accordance with the present disclosure, together with the bushing, securement mechanism, burr guard and burr shown in FIG. 9; and

FIG. 14 is a perspective view of another alternative conical burr template made in accordance with the present disclosure, together with the bushing, securement mechanism, burr guard and burr shown in FIG. 9.

Corresponding reference characters indicate corresponding parts throughout the several views. The exemplifications set out herein illustrate exemplary embodiments of the present invention, and such exemplifications are not to be construed as limiting the scope of the invention in any manner.

### DETAILED DESCRIPTION

The present disclosure provides burr template which has an irregular conical shape corresponding to a similarly shaped tibial cone augment, such that the template may be used as a guide track for a burr to quickly and accurately form a cavity in the tibia sized to correspond to the tibial cone shaped augment. As described in detail below, the burr template references the intramedullary canal of the tibia, thereby ensuring that the cone-shaped void created by using the burr template has a desired geometrical and spatial relationship with the anatomic intramedullary canal (and, therefore, with the other anatomic shapes and features of the natural tibia).

Although the exemplary embodiment described herein is adapted for use in conjunction with cone-shaped tibial augment components, it is contemplated that any template shape and size may be provided for use with any augment configuration, including cylindrical or other geometries, such as for other bones in human and animal anatomy. Further, although the exemplary template described herein is referred to as a “burr template” because a burr is an exemplary cutting tool used in conjunction with the template, it is contemplated that any suitable cutting tool may be used in conjunction with the present template as desired or required for a particular application.

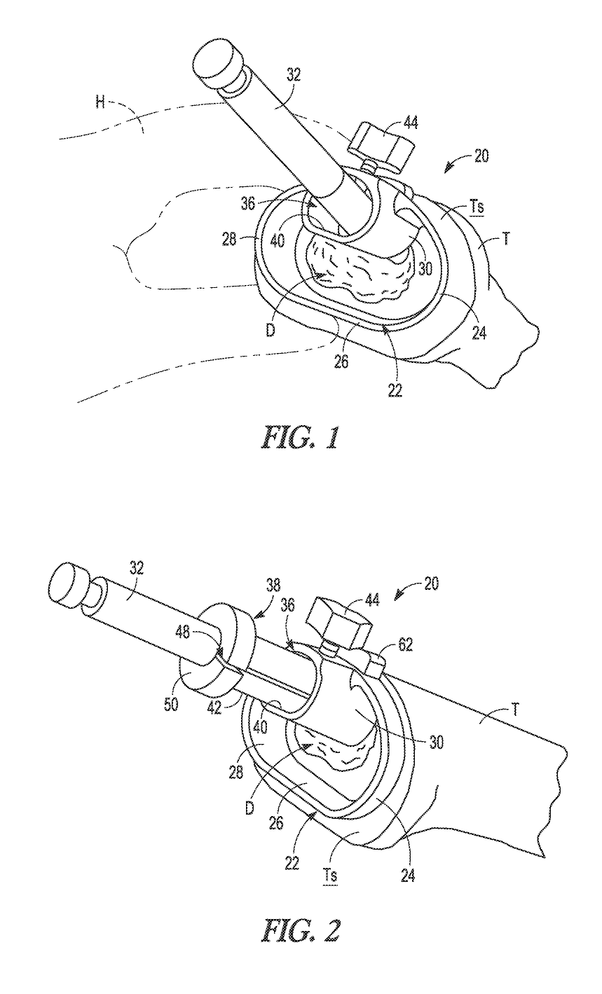

Turning now to FIG. 1, conical burr template 20 is shown positioned adjacent tibia T by surgeon's hand H. Template 20 includes an arcuate template track 22 having lateral track portion 24, posterior track portion 26, and medial track portion 28. Lateral and medial track portions 24, 28 are joined at the anterior side of template 20 by coupler 30, which is operable to couple template 20 to intramedullary rod 32 at desired a position and orientation (as described in detail below).

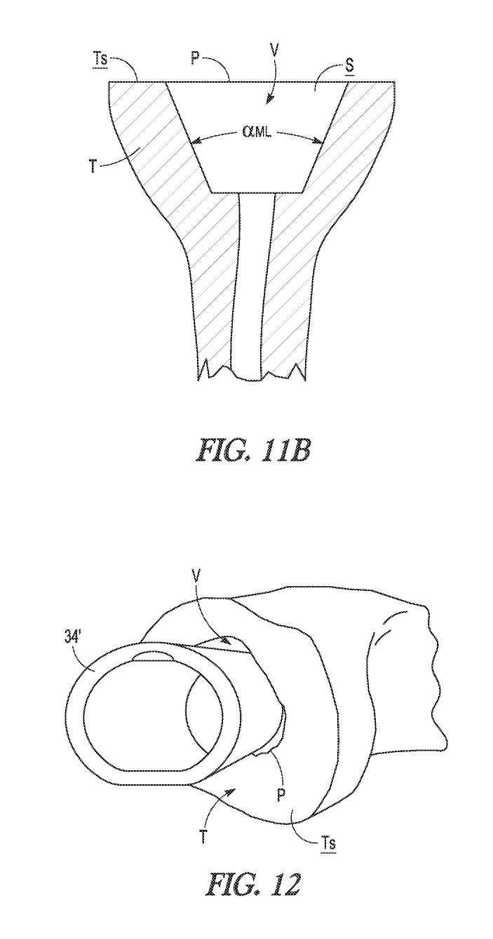

Conical burr template 20 is sized to substantially encompass bone defect D in the metaphyseal and/or diaphyseal region of tibia T, as illustrated in FIG. 1. In order to accommodate a variety of bone sizes and varying geometries of bone defect D, a kit of conical burr templates similar to burr template 20 may be provided, in which each different template has a different overall size and/or geometrical configuration from each other template in the kit. Moreover, each template provided in the kit may be specifically sized and shaped to yield a resection void V (FIGS. 11 and 12) which specifically fits an existing tibial cone augment size and shape.

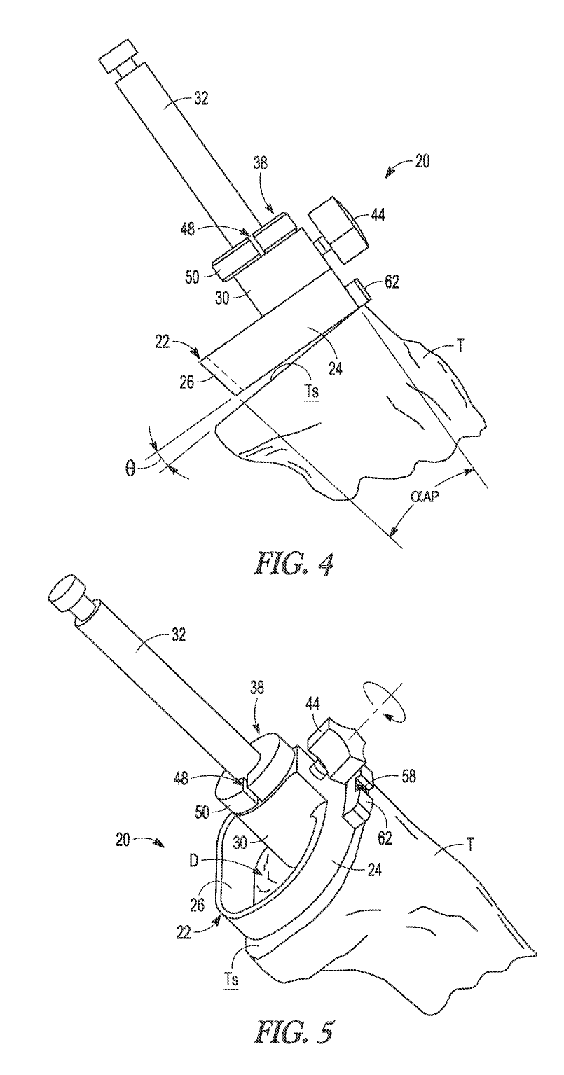

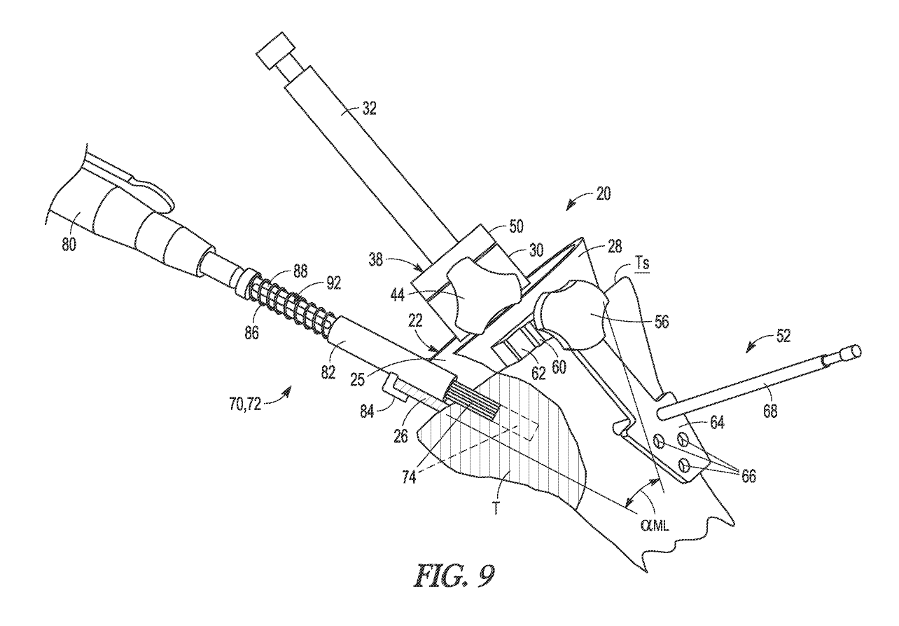

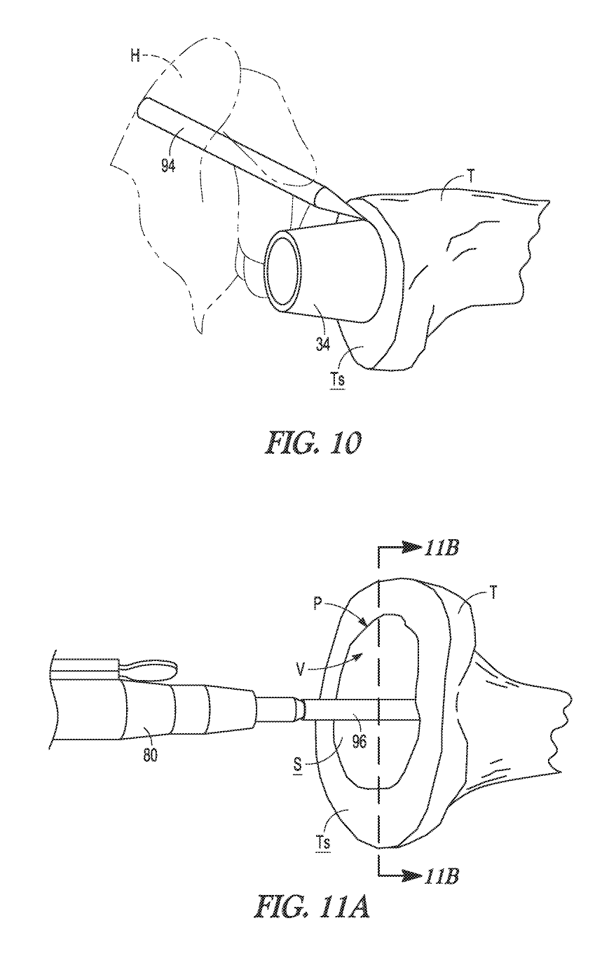

For example, burr template 20 defines anteroposterior taper α_{AP} (FIG. 4) between posterior track portion 26 and an anterior face of coupler 30, and medial-lateral taper α_{ML} (FIG. 9) between lateral track portion 24 and medial track portion 28. Taper angles α_{AP} and α_{ML} correspond with the analogous taper angles on a particular tibial cone augment, such as augments 34, 34′ (FIGS. 10 and 12). Therefore, after burr template 20 is used to create resection void V as described in detail below, resection void V is sized and shaped to accept tibial cone augment 34. Exemplary tibial augments, and geometrical details thereof, are disclosed in U.S. patent application Ser. Nos. 11/560,276, 12/886,297 and 61/488,549, incorporated by reference above.

Before template 20 is placed adjacent tibia T as shown in FIG. 1, tibia T is prepared in accordance with conventional arthroplasty procedures. For example, tibia T may have its proximal surface resected to create a generally planar proximal tibial surface T_{S}. In addition, the intramedullary canal of tibia T (not shown) may be reamed to accept intramedullary rod 32, such that the longitudinal axis of intramedullary rod 32 is generally coaxial with the longitudinal axis of the intramedullary canal (and, therefore, of tibia T). With tibia T thus prepared, the size and extent of bone defect D may be measured or estimated in order to select an appropriate conical burr template from a provided kit (described above), or a plurality of templates may be overlaid on bone defect D for a visual estimation.

Coupler 30 includes a coupler bore 36, which is passed over intramedullary rod 32 to bring template 20 adjacent to resected surface T_{S} of tibia T. As illustrated in FIG. 1, coupler bore 36 is oversized, such that bore 36 provides an easy clearance fit over intramedullary rod 32. Alignment bushing 38 is then passed over intramedullary rod 32 and into bore 36, as shown in FIG. 2. The clearance between bore 36 and rod 32 provides a space for receipt of alignment bushing 38, such that when alignment bushing 38 is received within bore 36, the clearance is substantially consumed and template 20 is substantially immovable in an anteroposterior or medial-lateral direction with respect to tibia T.

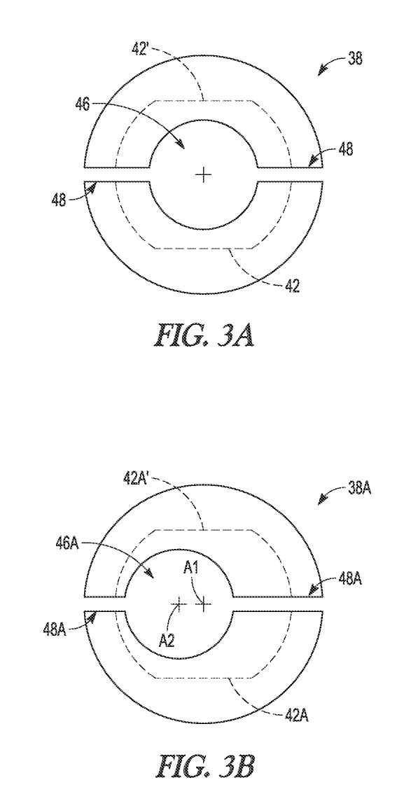

Coupler bore 36 further includes flat 40 formed therein, which corresponds to flat 42 of alignment bushing 38 (FIG. 3A). Flats 40, 42 cooperate to prevent rotation of template 20 with respect to the longitudinal axis of intramedullary rod 32 once template 20, bushing 38, and rod 32 are all affixed to one another (using set screw 44, as shown in FIG. 5 and described in detail below).

Turning to FIG. 3A, bushing 38 includes bore 46 sized to mount to intramedullary rod 32 (FIG. 2). In the illustrated embodiment, alignment bushing 38 is provided as two halves, such that bore 46 is created by joining the two halves as shown. When bore 36 is occupied by rod 32, gap 48 remains between the two halves of alignment bushing 38.

Turning to FIGS. 4 and 5, alignment bushing 38 is shown fully received within coupler bore 36. When so received, flange 50 of bushing 38 abuts the upper or proximal face of coupler 30. To affix alignment bushing 38 and template 20 to intramedullary rod 32, set screw 44 is tightened as best shown in FIG. 5. More particularly, threaded engagement between set screw 44 and coupler 30 drives set screw 44 into flat 42′ (FIG. 3A), which in turn applies pressure onto flat 42′ to capture intramedullary rod 30 between the halves of alignment bushing 38. As this pressure is applied, gaps 48 narrow slightly and flat 42 of bushing 38 is urged against flat 40 of bore 36. The pressure generates friction between bore 46 and intramedullary rod 32, and between coupler 30 and coupler bore 36. This friction effectively fixes template 20, bushing 38, and intramedullary rod 32 to one another. Because intramedullary rod 32 is substantially immovably fixed to tibia T, template 20 is also immovably fixed to tibia T.

As noted above, bushing 38 occupies the clearance space between intramedullary rod 32 and coupler bore 36, such that bushing 38 cooperates with the geometry of template 20 to fully constrain the spatial location and orientation of template 20 with respect to intramedullary rod 32 (and, therefore, also with respect to tibia T). In some cases, centered bushing 38 also centers template 20 over tibia T. However, in other cases, a patient's natural intramedullary canal is not centered with respect to the geometry of the proximal tibia T, which leads to an off-center orientation of template T. In such cases, it may be appropriate to offset template 20 with respect to intramedullary rod 32.

To accommodate such offset, offset alignment bushing 38A (FIG. 3B) may be provided. Offset bushing 38A is similar to centered alignment bushing 38 described above, and reference numbers in FIG. 3B refer to analogous structures shown in FIG. 3A and described above with respect to bushing 38. However, bore 46A defines longitudinal axis A₂ which is offset with respect to longitudinal axis A₁ of bushing 38A. In the illustrative embodiment of FIG. 3B, the direction of this offset is generally parallel to flats 42A, 42A′, such that using offset bushing 38A will laterally or medially offset template 20 (depending on which way bushing 38A is installed in bore 36). It is contemplated that a similar offset may also be accomplished in an anterior or posterior direction by positioning bore 46A closer to one of flats 42A, 42A′.

Turning now to FIG. 4, burr template 20 can also be used when proximal tibial surface T_{S} is angled with respect to the longitudinal axis of intramedullary rod 32. For example, in some surgical procedures it may be desirable to impart an anteroposterior slope to proximal tibial surface T_{S}, such as to accommodate particular prosthesis design or to correct for natural deformity. In the illustrative embodiment of FIG. 4, a positive anterior slope having slope angle Θ has been provided, i.e., proximal tibial surface T_{S} elevates or “runs uphill” with respect to the longitudinal axis of intramedullary rod 32 as one traverses from the posterior portion of the tibia T is toward the anterior portion of tibia T. In addition to anteroposterior slope, a non-planar surface T_{S} may be provided, or medial-lateral slope may be used (such as to correct for varus or valgus deformity), or any combination thereof. Advantageously, template 20 is connected directly to intramedullary rod 32, and is therefore indexed only to the longitudinal axis of intramedullary rod 32 (and, therefore, to the intramedullary canal of the tibia T) without regard to the geometry of proximal tibial surface T_{S}. Thus, any slope or other geometry may be made on proximal tibial surface T_{S} without disturbing the intramedullary referencing of template 20.

In some instances, intramedullary rod 32 may not be pinned or otherwise fixed to tibia T. While intramedullary rod 32 will typically define a close tolerance fit with the reamed intramedullary canal of tibia T, intramedullary rod 32 may remain axially movable and rotatable about its longitudinal axis throughout knee replacement surgery. In order to prevent corresponding rotation and/or axial movement of template 20, it may be desirable to provide a secondary fixation of template 20 to tibia T.

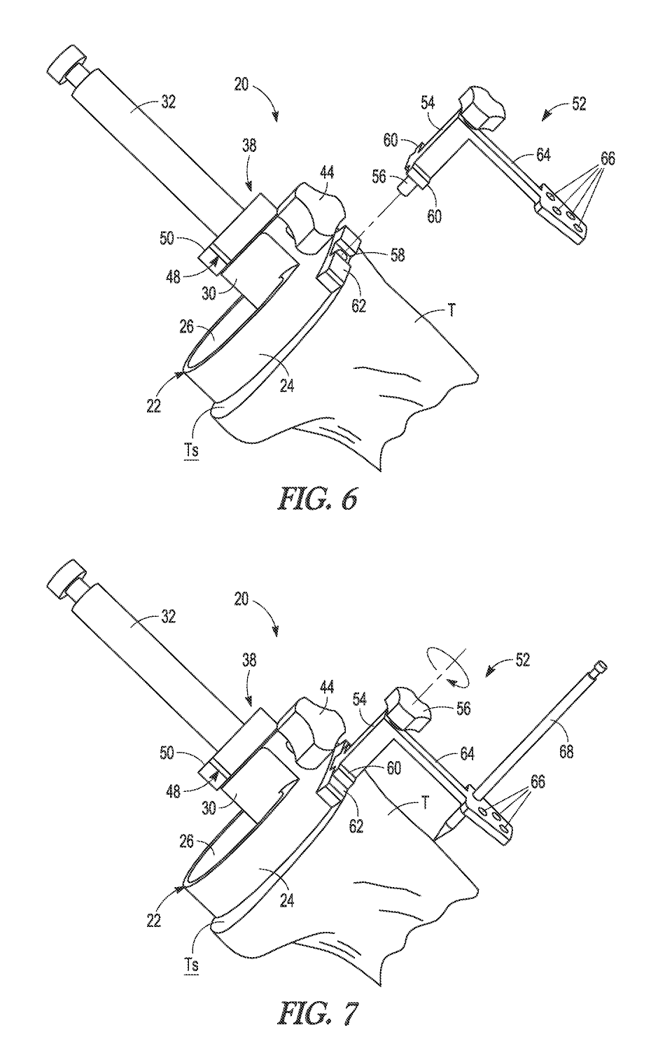

Turning to FIG. 6, securement mechanism 52 may be provided to facilitate such fixation. Securement mechanism 52 includes anteroposteriorly extending arm 54 with set screw 56 extending therethrough. Set screw 56 is received in threaded bore 58 formed in an anterior portion of template 20, and threaded engagement between set screw 56 and bore 58 fixes securement mechanism 52 to template 20 as shown in FIG. 7. In order to facilitate proper alignment of securement mechanism 52 with template 20, shoulders 60 may be provided to engage a correspondingly shaped outer face 62 adjacent threaded bore 58, as shown in FIG. 6. Fixation arm 64 extends distally from anteroposterior arm 54, and includes a plurality of apertures 66 therethrough. Referring to FIG. 7, pin 68 may be passed through one or more apertures 66 and into tibia T to provide additional fixation of template 20.

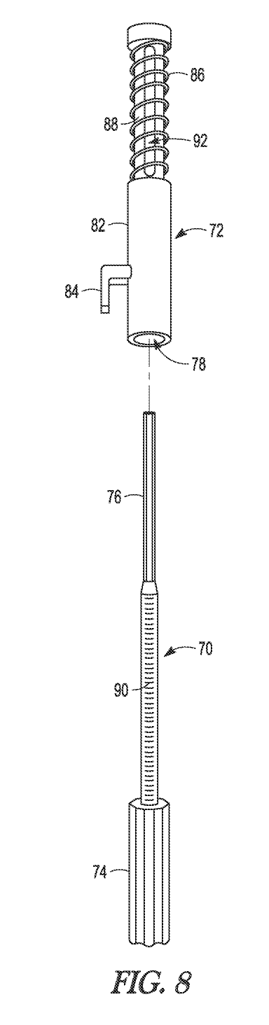

With template 20 firmly affixed to tibia T, template track 22 may be used to define the perimeter of resection void V (FIGS. 10 and 11). Referring to FIG. 8, the cutting tool used to define such perimeter includes burr 70, which is received within burr guard 72. Specifically, burr 70 includes cutting head 74 and drive shaft 76, with drive shaft 76 received within bore 78 of burr guard 72. When connected to rotary tool 80, as shown in FIG. 9, burr 70 passes through burr guard 72 and into the bone stock of tibia T as described below. Burr guard 72 includes outer tube 82 with arm 84 extending therefrom. Arm 84 engages template track 22 as shown in FIG. 9 to maintain cutting head 74 at particular angular orientation with respect to the longitudinal axis of intramedullary rod 32 as burr guard 72 is swept around the periphery of template track 22 from lateral track portion 24, through posterior track portion 26 and to medial track portion 28 (or vice versa).

As burr guard 72 is swept around the periphery of template track 22, cutting head 74 can be plunged into tibia T by pushing on rotary tool 80 against the bias of spring 86 as shown in FIG. 9 Inner tube 88 is received within outer tube 82, and is axially movable within bore 78 to allow cutting head 74 to be selectively moved between a withdrawn position and a projected position. In the withdrawn position, cutting head 74 is received completely within bore 78 of outer tube 82, such that the cutting head is incapable of effecting any resection. In the projected position, cutting head 74 projects outwardly from outer tube 82, such that cutting head can resect material upon contact. Spring 86 urges cutting head into the fully withdrawn, stowed position in the absence of affirmative applied force overcoming such bias, such that outer tube 82 provides a protective sheath for cutting head 74. Advantageously, this “normally withdrawn” configuration ensures that cutting head 74 is only exposed when the user engages burr guard 72 with template track 22 and then actively exerts an axial force on rotary tool 80, as described below.

A surgeon begins the resection process by engaging arm 84 of burr guard 72 with guide track 22. Once arm 84 is so engaged, the surgeon applies a downward axial force to rotary tool 80, which compresses spring 86 and causes cutting head 74 to emerge from within bore 78 of outer tube 82. Cutting head 74 is then engaged with tibia T to begin the resection process. Cutting head 74 is then plunged to a specified depth, as shown in dashed lines in FIG. 9. Advantageously, cutting head 74 will automatically retract into outer tube 82 if the axial force is removed for any reason, such as upon completion of the resection operation or if arm 84 becomes inadvertently disengaged from guide track 22.

Resection continues by sweeping burr guard 72 around track 22, while keeping arm 84 engaged with guide track 22. At each new position, cutting head 74 is successively plunged into tibia T to the specified depth. Once cutting head has been plunged at each position around guide track 22, a lateral, posterior, and medial periphery corresponding to tibial cone augments 34, 34′ (FIGS. 10 and 12) is formed in tibia T. Pin 68 and intramedullary rod 32 may then be removed from tibia T, together with template 20 and securement mechanism 52, leaving the medial, posterior and lateral portions of the resection periphery exposed.

During the resection process, the depth of resection may be monitored by depth markings 90 on drive shaft 76, which are visible through cut-out 92 formed in inner tube 88. Cutting head 74 may be plunged to the full intended depth at each position on the first sweep around guide track 22, such that the guide-track resection process is complete after single sweep. Alternatively, cutting head 74 may be plunged to a partial depth initially and to deeper depths in one or more subsequent sweeps around guide track 22 until the desired resection depth is achieved.

Due to the presence of coupler 30, the anterior portion of periphery P of resection void V (FIGS. 11A and 11B) is not defined through the use of template 20. To define this portion of periphery P after removal of template 20, tibial cone augment 34 may be placed upside down, as shown in FIG. 10, over the medial, posterior and lateral periphery created by the sweep-and-plunge resection process described above. Augment 34 is positioned on tibial surface T_{S} such that the lateral, posterior, and medial portions of tibial cone augment 34 are aligned with their corresponding lateral, posterior, and medial peripheral cuts made in tibia T. In an exemplary embodiment, tibial cone augment 34 used for this step is a provisional tibial cone augment, though it is contemplated that permanent tibial cone augment, such as augment 34′ (FIG. 12) may also be used.

With the proximal space of tibial cone augment 34 properly positioned upside down on proximal surface T_{S} of tibia T the anterior portion of periphery P (FIG. 11A) may be traced around tibial cone augment 34 with marking instrument 94, such as manually by surgeon hand H as shown in FIG. 10. With the proper periphery thus marked, the remainder of periphery P may be created by freehand resection with any suitable tool, such as burr 72 described above. Coupler 32 may be maintained at a minimal size and extent within burr template 20, which minimized the extent of the anterior freehand resection.

Referring still to FIGS. 11A and 11B, the remainder of the interior bone within the periphery P defined by the above described resection process may be removed to create resection void V. In an exemplary embodiment, this removal is conducted with the aid of burr sleeve 96, which prevents the precisely formed periphery P and inner surface S of resection void V from being disturbed during the removal of the remaining interior bone. When this interior bone is fully removed to proper depth, resection void V is complete.

Finally, as shown in FIG. 12, permanent tibial cone augment 34′ may be press-fit into resection void V. Ideally, such fit will be tight but will pose no risk for damage to tibia T by being too tight. Although a surgeon may make additional small resections at the margins of periphery P and/or resected surface S, such corrections are minimized or eliminated by the high degree of accuracy and precision afforded by template 20 and the associated components and methods described herein.

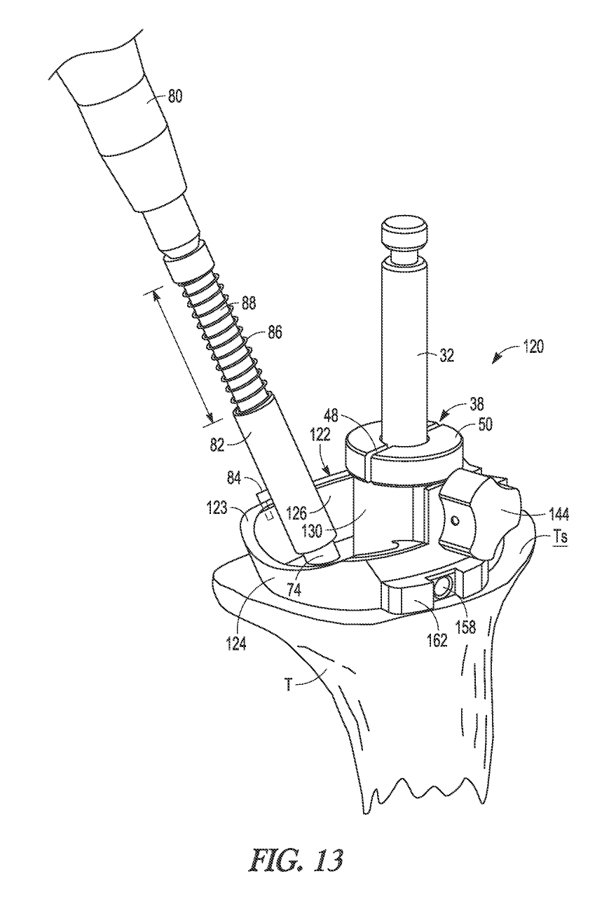

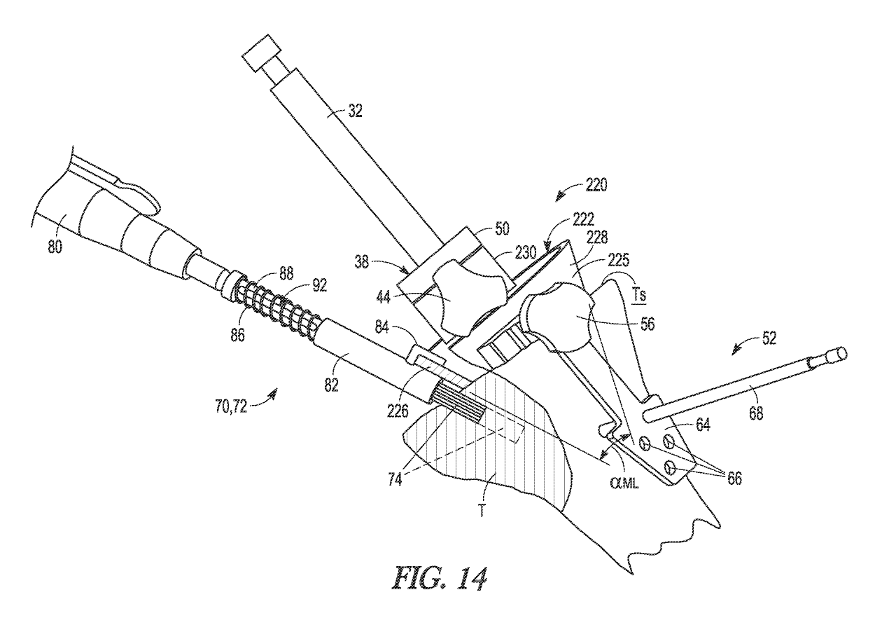

Turning now to FIG. 13, an alternative embodiment is shown in which the depth of resection of periphery P is controlled by the upper face of a conical burr template. Rather than monitoring resection depth by depth markings 90 of burr 70 (FIG. 8), resection depth is controlled by contact between burr guard 72 and proximal face 123 of template track 122 of burr template 120, as described in detail below. Burr template 120 is similar to burr template 20 described above, and reference numbers in FIG. 13 refer to analogous structures shown in FIG. 1-9 and described above with respect to template 20. However, template 120 includes a “rollercoaster”-like upper face 123 which defines variable height above resected surface T_{S} of tibia T. This variable height compensates for changes in the taper angles between various portions of template track 122, thereby allowing the user to achieve a constant resection depth with respect to the longitudinal axis of intramedullary rod 32 without monitoring depth markings 90.

In the exemplary embodiment of FIGS. 1-9, medial lateral taper angle α_{ML} (FIG. 9) is larger than anteroposterior taper angle α_{AP} (FIG. 4). As cutting head 74 of burr 70 is swept around guide track 20 keeping arm 84 in contact therewith, cutting head 74 must be plunged relatively more deeply into tibia T at areas of high angulation (such as angle α_{ML} at lateral and medial track portions 24, 28) as compared with areas of lower angulation (such as angle α_{AP} at posterior track portions 26). The depth of plunge can be calculated for the different track portion angles to compensate for the additional axial, linear distance that must be traversed by cutting head 74 in the high-angulation areas, thereby producing a constant overall resection depth with respect to tibial surface T_{S}. This calculated depth can then monitored by depth markings 90 as noted above.

However, the embodiment of FIG. 13 is configured to automatically adjust the plunge depth to accommodate varying track portion angles. Posterior track portion 126, which defines a relatively smaller taper angle with respect to tibial surface T_{S}, defines relatively elevated or “tall” portion of proximal face 123, i.e., a portion set relatively far away from tibial surface T_{S}. As template track transitions to lateral portion 124, which defines relatively larger taper angle with respect to tibial surface T_{S} proximal face 123 slopes downwardly toward tibial surface T_{S} to define a relatively depressed or “short” portion of proximal face 123, i.e., a portion set relatively close to tibial surface T_{S}. Medial portion 126 (not shown) may be similarly configured with a dip or depression suitable for its particular angulation.

In the embodiment of FIG. 13, cutting head 74 is limited to a fixed axial travel and is extended to the full extent of such axial travel throughout the resection process. For example, cutting head 74 may be advanced only up to a fixed amount before encountering a physical barrier to further advance, such a fully compressed spring 86. At each position around template track 122 during the resection process, cutting head 74 may extend outwardly from outer tube 82 (i.e., “plunged”) and into tibia T by the same linear, axial amount. Assuming arm 84 remains in constant contact with template track 122, this constant axial travel of cutting head 74 cooperates with the variable height of proximal face 123 to produce a constant resection depth around the entire periphery of template track 122.

Turning again to FIG. 9, it can be seen that burr guard 72 and burr 70 pass through template 20 along inner face 25 of template track 22. As shown in FIG. 14, however, burr template 220 may be provided which facilitates the tracing of burr 70 and burr guard 72 around exterior face 225 of template track 222. Burr template 220 is similar to burr template 20 described above, and reference numbers in FIG. 14 refer to analogous structures shown in FIG. 1-9 and described above with respect to template 20. However, template track 222 defines a smaller circumference as compared to template track 22, such that tracing template track 222 around exterior face 225 produces the same resection void V produced by tracing interior face 25 of template track 22.

Advantageously, this “exterior tracing” modality allows conical burr template 220 to be made smaller than burr template 20 for a given size and geometry of resection void V. Further, tracing exterior face 225 allows greater visual access to cutting head 74 during the resection procedure, because cutting head 74 is fully exposed to the surgeon rather than contained within the template track.

Template track 222 may also be used in the same manner as template track 22, i.e., with burr 70 and burr guard 72 traced around the inner face of track 222, or vice-versa. For example, a surgeon may choose to sweep around the exterior of guide track 22 to convert resection void V from being sized for press-fit engagement with tibial cone augment 34′, as detailed above, to being sized for a clearance fit suitable for use with cemented fixation of augment 34′.

It is contemplated that the wall thickness of the template track (e.g., the thickness along direction normal to the longitudinal axis of intramedullary rod 32 in template tracks 22, 222) may be varied to change the difference in size of the associated resection void. For example, relatively thick template track wall will result in relatively large difference in such sizes, such that one template may be suitable for two different implant sizes. In this example, the smaller implant would correspond to the resection void created by sweeping a cutting instrument around the inner face of the track, while the larger implant would correspond to the resection void created by sweeping the cutting instrument around the outer face of the track. On the other hand, relatively thin template track might be appropriate for defining press-fit and clearance fits, as described above

Advantageously, the method and apparatus disclosed herein facilitate the creation of a resection cavity sized and shaped for an improved fit between the cavity and augment.

While this invention has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. An apparatus comprising:
a burr template including:
a template track including a lateral track portion, a posterior track portion, and a medial track portion; and
a coupler joining the lateral track portion and the medial track portion, the coupler including a bore formed therethrough, the bore being spaced from the lateral track portion and the medial track portion, wherein the template track defines an inner periphery corresponding to an outer periphery of a tibial cone augment, the inner periphery configured to be indexed to an intramedullary canal of a tibia.

2. The apparatus of claim 1, comprising:
a burr assembly including:
a burr including a cutting head adapted to resect bone; and
a burr guard including a tube sized to receive the cutting head of the burr, the cutting head being axially moveable within the tube between a withdrawn position and a projecting position, the burr being completely received within the tube in the withdrawn position.

3. The apparatus of claim 2, wherein the tube includes an arm sized to engage with the template track, wherein the cutting head is configured to be urged from the withdrawn position toward the projecting position with engagement of the arm with the template track and application of an axial force to the burr.

4. The apparatus of claim 1, wherein the bore of the coupler is configured to accept an intermedullary rod disposed within the intramedullary canal of the tibia, the coupler being engageable with the intermedullary rod to position the burr template with respect to the tibia.

5. The apparatus of claim 4, wherein the burr template includes an alignment bushing engageable within the bore of the coupler, the alignment bushing including an opening configured to accept the intermedullary rod and align the intermedullary rod with respect to the burr template.

6. The apparatus of claim 5, wherein the opening of the alignment bushing is substantially centered with respect to the alignment bushing.

7. The apparatus of claim 5, wherein the opening of the alignment bushing is offset from a center of the alignment bushing.

8. The apparatus of claim 1, wherein the burr template includes a securement mechanism engageable with the burr template, the securement mechanism including a fixation arm engageable with the tibia, the securement mechanism being configured to affix the burr template with respect to the tibia.

9. The apparatus of claim 8, wherein the fixation arm includes a pin extending from the fixation arm and engageable with the tibia for engagement of the fixation arm with the tibia.

10. The apparatus of claim 1, wherein the burr template defines an anteroposterior taper between the posterior track portion and a face of the coupler.

11. The apparatus of claim 1, wherein the burr template defines a medial-lateral taper between the lateral track portion and the medial track portion.

12. The apparatus of claim 1, wherein the template track includes an upper face including a height above a resected surface of the tibia, the height varying along the template track.

13. A method for resecting a cavity in a bone, the method comprising:
inserting an intramedullary rod into an intramedullary canal of the bone;
passing a coupler of a template over the intramedullary rod and coupling the template to the intramedullary rod adjacent the bone, the template including a template track extending away from the coupler; and
moving a cutting instrument around the template track to define an inner periphery of a resection void.

14. The method of claim 13, wherein coupling the template to the intramedullary rod includes placing an alignment bushing within a bore of the coupler, the alignment bushing including an opening configured to accept the intermedullary rod and to align the intermedullary rod with respect to the template.

15. The method of claim 14, comprising selecting the alignment bushing, wherein the opening of the alignment bushing is substantially centered with respect to the alignment bushing.

16. The method of claim 14, comprising selecting the alignment bushing, wherein the opening of the alignment bushing is offset from a center of the alignment bushing.

17. The method of claim 13, comprising selecting the template from a plurality of templates, wherein each of the plurality of templates includes a size and shape to yield a resection void corresponding to one of a plurality of cone augment sizes and shapes.

18. The method of claim 13, wherein moving the cutting instrument around the template track includes moving the cutting instrument around an interior of the template track.

19. The method of claim 13, wherein moving the cutting instrument around the template track includes moving the cutting instrument around an exterior of the template track.

20. The method of claim 13, comprising fixating the template to the bone using a securement mechanism coupled to the template and engageable to the bone, the securement mechanism configured to inhibit at least one of rotation or axial movement of the template with respect to the tibia.
